(19) United States
(12) Patent Application Publication
(10) Pub. No.: US 2013/0340767 A1
(43) Pub. Date: Dec. 26, 2013

(54) Spring Biased Tourniquet Especially Suited for Use with a Peripherally Inserted Central Catheter

(71) Applicants: Velda L. Biddex, Marion, NC (US); Karen T. Johnson, Anderson, SC (US); Sandy Maney, Waynesville, NC (US); John David Guy, Winston-Salem, NC (US)

(72) Inventors: Velda L. Biddex, Marion, NC (US); Karen T. Johnson, Anderson, SC (US); Sandy Maney, Waynesville, NC (US); John David Guy, Winston-Salem, NC (US)

(21) Appl. No.: 14/011,007

(22) Filed: Aug. 27, 2013

Related U.S. Application Data
(63) Continuation-in-part of application No. 13/048,998, filed on Mar. 16, 2011.
(60) Provisional application No. 61/743,125, filed on Aug. 27, 2012.
(60) Provisional application No. 61/753,309, filed on Jan. 16, 2013.

(51) Int. Cl. A61B 17/132; A61B 19/08

(52) U.S. Cl. CPC A61B 17/1322; A61B 19/08
USPC 128/849; 606/203

(57) ABSTRACT

A spring biased tourniquet can be placed on the exterior of a patient's appendage, such as on his or her arm. The spring tourniquet will exert pressure on the appendage to constrict and artery or dilate vein. Terminal ends of the spring member can be pulled apart to either reduce pressure or to disengage the patient's appendage. This spring biased tourniquet is especially suited for use in a peripherally inserted central catheter (PICC) procedure where the tourniquet can be placed on the exterior of a surgical drape, where it remains visible, and pressure can be reduced to avoid thrombosis without compromising the sterile barrier surrounding the patient during this procedure.

### CROSS REFERENCE TO PRIOR CO-PENDING APPLICATIONS

This application is a continuation in part of co-pending U.S. patent application Ser. No. 13/048,998 filed on Mar. 16, 2011, entitled Quick Release Tourniquet and abandoned upon filing of this application. This application also claims the benefit of co-pending U.S. Provisional Patent Application 61/743,125 filed Aug. 27, 2012 and also claims the benefit of co-pending U.S. Provisional Patent Application 61/753,309 filed Jan. 16, 2013.

### BACKGROUND OF THE INVENTION

The present invention is directed to a tourniquet, which is suitable for use in multiple and diverse applications. More specifically, the present invention is related to a tourniquet, which allows for use in multiple applications and is quickly applied and released.

Tourniquets have been widely used in the medical arts for such applications as compressing an underlying blood vessel to securing a drape in the vicinity of a medical procedure site. There are a myriad of tourniquets from as simple as a flexible tube which is secured around the arm to complex bladder type tourniquets as commonly employed in a blood pressure cuff. Many tourniquets have closure mechanisms such as snaps, VELCRO®, various engaging members and the like which are cumbersome to use, especially while wearing gloves as is most often the case.

Of particular relevance to the present invention are tourniquets made from materials with spring type properties which can be manipulated to be placed on an appendage and then allowed to relax to a near rest shape. These have previously been considered inferior due to the inability to control the relaxation and they cannot be placed in a way to selectively collapse blood vessels or not collapse blood vessels.

There is an ongoing need for an improved tourniquet which is devoid of the deficiencies of the art. The present invention provides such a device which is easily manipulated for quick release.

### SUMMARY OF THE INVENTION

It is an object of the invention to provide a quick release tourniquet.

It is another object of the invention to provide an improved tourniquet which can be used in multiple and diverse applications.

One advantage of the inventive tourniquet is the ease of use.

These and other advantages, as will be realized, are provided in a tourniquet. In one embodiment, the tourniquet has a generally hexagonally shaped upper frame. The upper frame has a linear upper frame elements meeting at an upper apex. Linear side frame elements meet a linear upper frame element at an upper side apex. linear lower frame elements meets one side linear side frame element at a lower side apex wherein the linear lower frame elements extend beyond an overlapping apex. Each linear lower frame element of said linear lower frame elements comprises a terminus with a handle attached to each terminus.

Yet another embodiment is provided in a method of applying a tourniquet to an appendage. The method includes providing a tourniquet wherein the tourniquet has a generally geometrically shaped frame with linear upper frame elements meeting at an upper apex; linear side frame elements wherein each linear side frame element of the linear side frame elements meets a linear upper frame element of the linear upper frame elements at an upper side apex; linear lower frame elements wherein each linear lower frame element of the linear lower frame elements meets one said side linear side frame element at a lower side apex each linear lower frame element of the linear lower frame elements each comprises a terminus; and handles wherein a handle of said handles is attached to each terminus. The method further includes grasping each handle and persuading the handles away from each other thereby forming a passage between the terminus sufficiently for the appendage to pass through the passage. The appendage is passed through the passage. The handles are allowed to decrease the passage to secure the appendage within the generally geometrically shaped upper frame. Pressure on the appendage or a vein or artery therein could also be reduced in this manner as part of a surgical procedure to prevent thrombosis.

The present invention is related to an improved tourniquet. More specifically the present invention is related to a tourniquet that can be easily released or removed from an appendage of a patient without requiring manipulation of mating components.

### BRIEF DESCRIPTION OF THE DRAWINGS

FIG. 1 is a schematic representation of a first embodiment of the invention in which terminal ends overlap.

FIG. 2 is a front schematic view of an embodiment of the invention.

FIG. 3 is a side schematic view of the embodiment shown in FIG. 2.

FIGS. 4A-4D illustrate an embodiment of the invention in schematic form.

FIG. 5 is a view of an alternate embodiment of the spring tourniquet of this invention in which the terminal ends do not overlap.

FIG. 6 is a view of another version of the invention similar to that shown in FIG. 5.

FIG. 7 is a view of another version of this invention in which the handles do not form a loop.

FIG. 8 is a view of still another version of the invention in which the top is curved.

FIG. 9 is a view of a version similar to FIG. 8 in which the handles are formed by straight sections.

FIG. 10 is a view of an embodiment similar to that shown in FIG. 9 but in which the top is flat.

FIG. 11 is a view of a semicircular version of this invention.

FIG. 12 is a view of an embodiment combining a semicircular spring member and a flexible fabric tourniquet member to apply pressure to a patient's appendage.

FIG. 13 is a view showing a scissors arrangement for either applying or relieving pressure depending upon the detailed construction of this version.

FIG. 14 is a view showing a prior art full body for maximal barrier protection of the type that is typically used in a peripherally inserted central catheter (PICC) procedure.

### DETAILED DESCRIPTION OF THE PREFERRED EMBODIMENTS

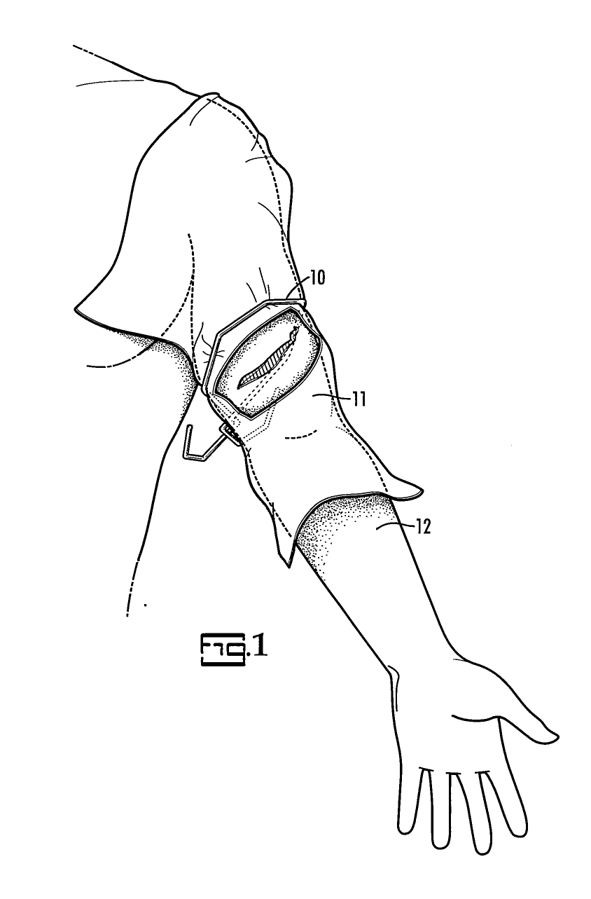

An embodiment of the invention will be described with reference to FIG. 1 wherein a tourniquet, 10, is shown around an appendage, 12, represented as a human arm. While Illustrated as an arm the appendage could be an arm, leg, thumb, finger or toe. In one embodiment the tourniquet can be used to secure a surgical drape, 11, in proper position during a surgical procedure. As will subsequently be discussed in more detail, the termini or terminal ends of the tourniquet 10, which contain handles 32, 32′ merely overlap, and the handles need not be twisted around the opposite ends of the tourniquet, and careful examination of FIG. 1 will show that the opposite ends of the tourniquet are not intertwined, but will remain in the position shown in FIG. 1 because of the spring characteristics of the material from which the tourniquet 10 is formed. In the embodiment of FIGS. 1-4A-4D, as well as in other embodiments, opposed handles or opposite ends of the tourniquets remain free from each other during their entire range of movement so that the spring characteristics of the tourniquet can be relied upon to exert pressure on the appendage and either close an artery or vein or dilate a vein. In this way, pressure can be applied to the appendage and to arteries or veins therein even though the size of the appendage may differ from one patient to another. Of course different size tourniquets 10 will typically be necessary for individuals of markedly different sizes, such as for example a child and a powerful athlete. Different tourniquet sizes would also be necessary for use with appendages of markedly different sizes, such as for example a forearm as compared to the same person's thigh.

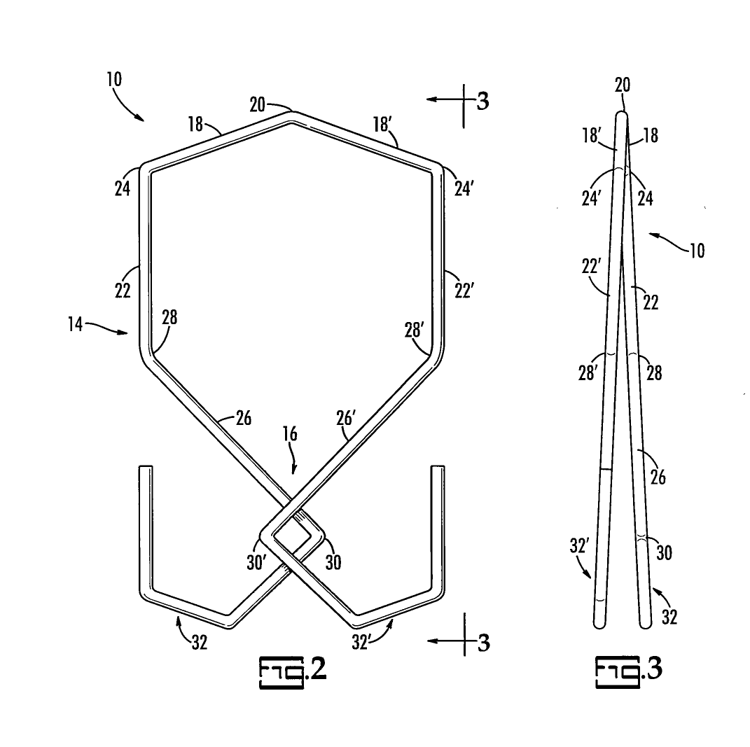

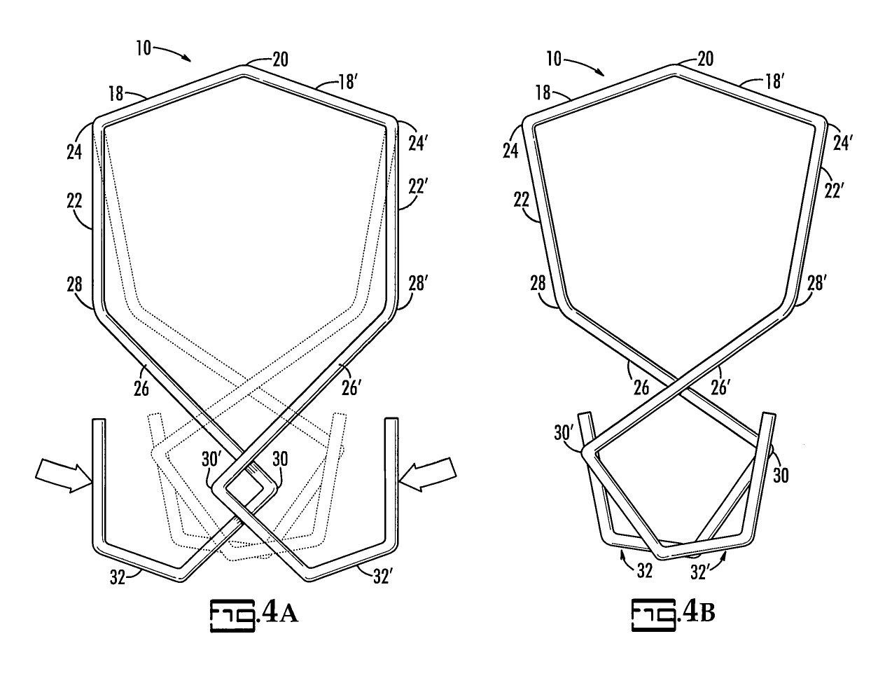

A tourniquet in accordance with an embodiment of the invention is illustrated in front view in FIG. 2 and FIG. 3 is side view, taken along line 3-3 of FIG. 2. The tourniquet, 10, comprises a generally hexagonally shaped upper frame 14, with an overlapping apex 16, which is not connected. The generally hexagonally shaped upper frame comprises linear upper frame elements, 18 and 18, meeting at an upper apex 20. Linear side frame elements 22 and 22′, each meets the linear upper frame elements 18 and 18′, at an upper side apex 24 and 24′. Linear lower frame elements 26 and 26′, each meet the linear side frame elements 22 and 22 at a lower side apex 28 and 28′. The linear lower frame elements 26 and 26′ extend beyond the overlapping apex 16 to a terminus or terminal end 30 and 30′.

Handles, 32 and 32, are attached to the terminus of the linear lower frame elements, 26 and 26′. The purpose of the handles will be further understood from disclosure herein. The handles preferably comprise linear portions generally in the shape of a vacated or open pentagon or vacated circle or loop wherein the term “vacated” indicates one linear portion, in the case of a pentagon, or one arc, in the case of a circle is absent. A generally pentagon shape is preferred due to simplicity in manufacturing. In one embodiment the handle may be continuous and integral to the furthest extent of the handle to the linear lower frame elements 26 and 26′. A continuous or closed handle may require additional manufacturing steps required to accomplish the attachment.

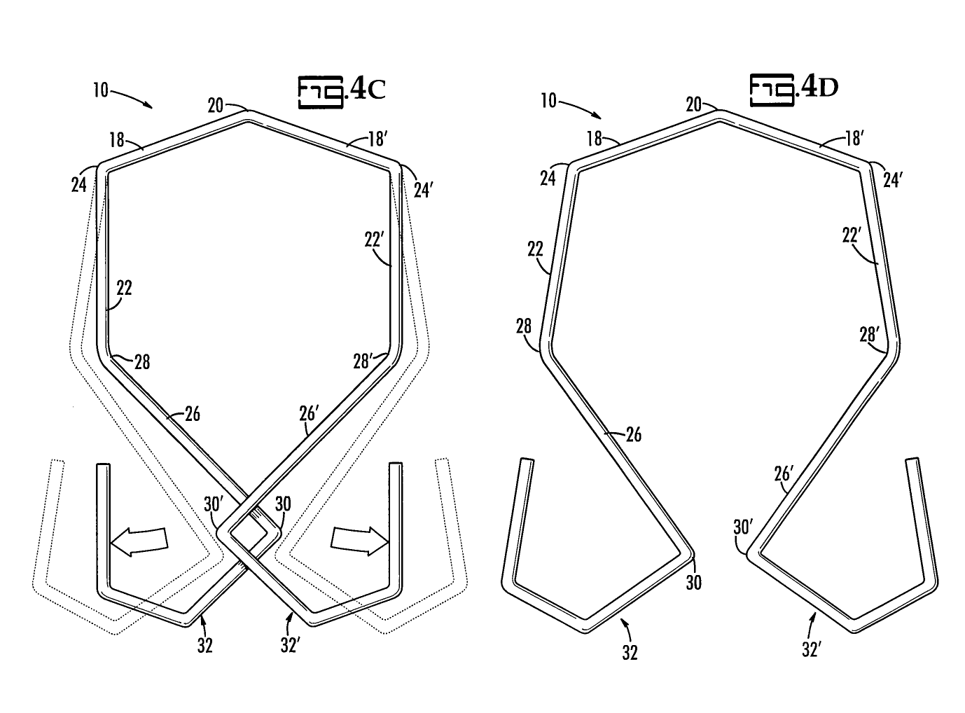

An embodiment of the invention will be described with reference to FIGS. 4A-4D. The tourniquet is illustrated in a rest shape in FIG. 48. In use, the technician will grasp the handles 32 and 32′, and draw the handles apart as represented by the arrows in FIG. 4C, thereby forming a passage 34, between the terminus 30 and 30′, of the linear lower frame elements, 26 and 26′. The handles are drawn apart to the extent necessary for the appendage to pass through the passage, 34, at which point the tourniquet is allowed to return towards rest shape as indicated by the arrows of FIG. 4A. As would be readily realized the extent to which the tourniquet relaxes towards rest shape is dependent on the size of the appendage contained therein.

An advantage of the tourniquet can be realized by examination of FIGS. 4A-4D. Round tourniquets may have a disadvantage in that when they are opened the entire ring is expanded evenly. Therefore, upon return to rest shape the ring changes size symmetrically. Potential advantages of round or semi-circular tourniquets will, however, be subsequently discussed with reference to other embodiments. With the first embodiment the present invention, the primary bending occurs at the lower side apex, 28 and 28′, and upper side apex, 24 and 24′, with minimal distortion at the upper apex, 20. This is advantageous, particularly when used with a drape, which is a cloth laid over at least a portion of the patient, since the drape can be held down by the linear upper frame elements. 18 and 18′, and the tourniquet can be allowed to relax without the drape becoming bunched under the tourniquet.

The linear upper frame elements, linear side frame elements and linear lower frame elements are independently at least 0.5 inches to 6 inches and more preferably at least 1 inch to 3 inches in length.

It is most preferred that the tourniquet is a continuous element bent into the shape as illustrated. Most preferably, the continuous element has a rounded cross-sectional shape although other cross-sectional shapes include square, pentagonal, hexagonal, etc. may be employed. A round cross-section is preferred due to the extensive availability of round materials which are suitable for use in the invention. Symmetrical shapes are preferred due to fact that symmetrical cross-sections are typically more readily extruded. In one embodiment the tourniquet comprises a central material with a non-allergenic coating thereon.

The material of construction is not particularly limiting with the proviso that the material has sufficient strength to maintain the basic shape when repeatedly distorted to place on the appendage and returns toward rest shape, as represented in FIG. 2 or 4B, when released. Rest shape is the shape which the tourniquet is in when distorting forces are not applied.

Geometrical shapes such as hexagon, pentagon, circular, etc. or their equivalent tenns such as hexagonal, pentagonal, round, circular, etc. are intended to include regular geometries and irregular geometries. Adjacent linear members may be the same length or a different length with the difference between adjacent linear members having a length which is at least 50% to no more than 150% relative to each other.

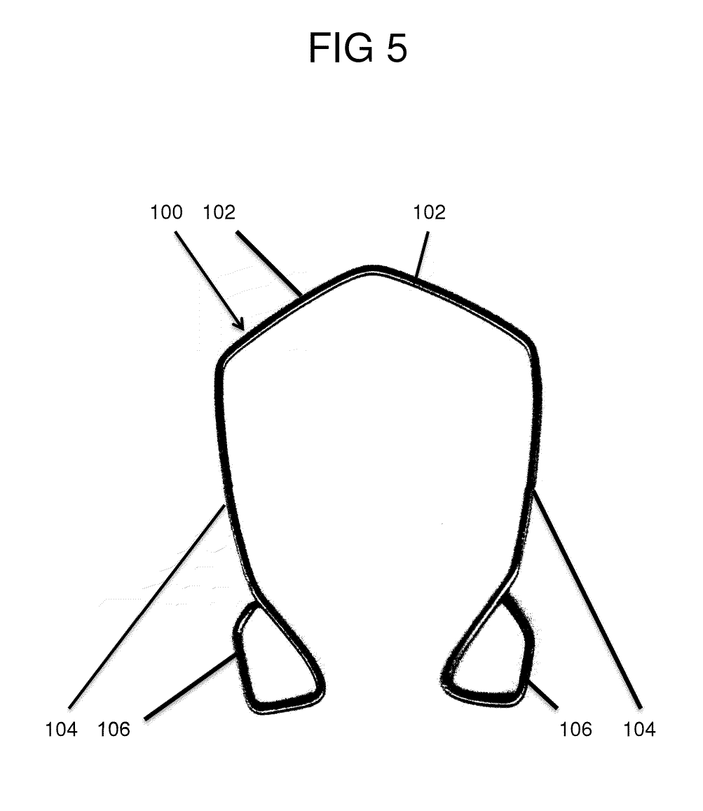

Alternate embodiments of tourniquets shown in FIGS. 5-13 are formed of materials having spring characteristics. The terminal ends or termini of these alternate embodiments do not overlap when the spring member is in the neutral or rest position or when they surround a patients appendage to apply pressure to that appendage or to arteries or veins therein.

FIG. 5 is a view of a generally hexagonal top open ended spring type tourniquet 100 with upper frame segments 102 and lower frame segments 104 with enclosed, but not overlapping, handles 106 that can be opened enough to place over an appendage that will spring back to properly compress the appendage to restrict blood flow for a procedure and that can be temporarily opened to permit blood flow during a procedure and that can be released to restrict blood flow during the procedure. The tourniquet 100 can also be removed during a procedure and replaced over the appendage when needed. The tourniquet 100 can be immediately removed after a procedure. The tourniquet 100 may also be used to hold a drape or other material in place as needed.

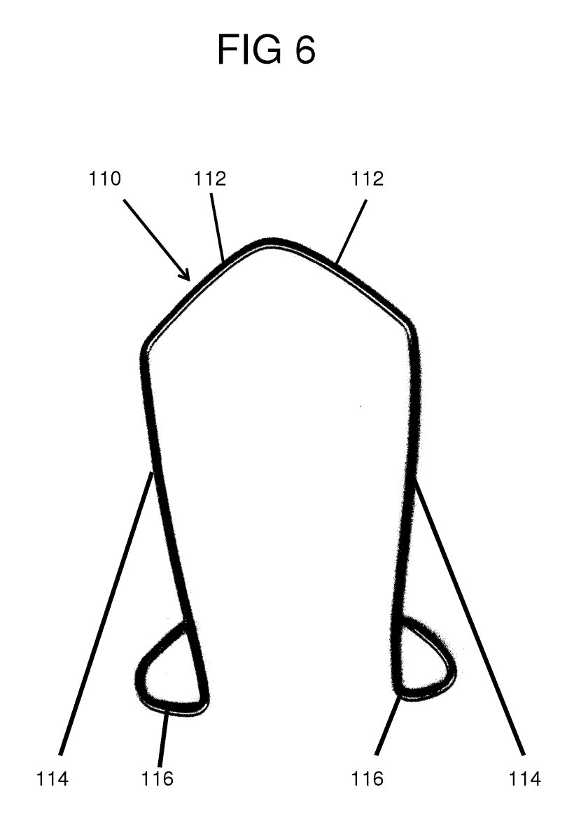

FIG. 6 is a view of a generally dome top spring type tourniquet 110 with upper frame segments 112 that are slanted inward with enclosed handle 116s at the lower end of the side frame segments 114 that can be sufficiently spread apart to place over an appendage and permitted to spring back to properly compress the appendage to restrict blood flow for a procedure. The tourniquet 110 can be temporarily spread apart to permit blood flow during a procedure and released to recompress the appendage as needed. Tourniquet 110 can also be removed and replaced as needed during a procedure and removed immediately after completion of the procedure. The tourniquet 110 may also be used to hold a drape or other material in place as needed.

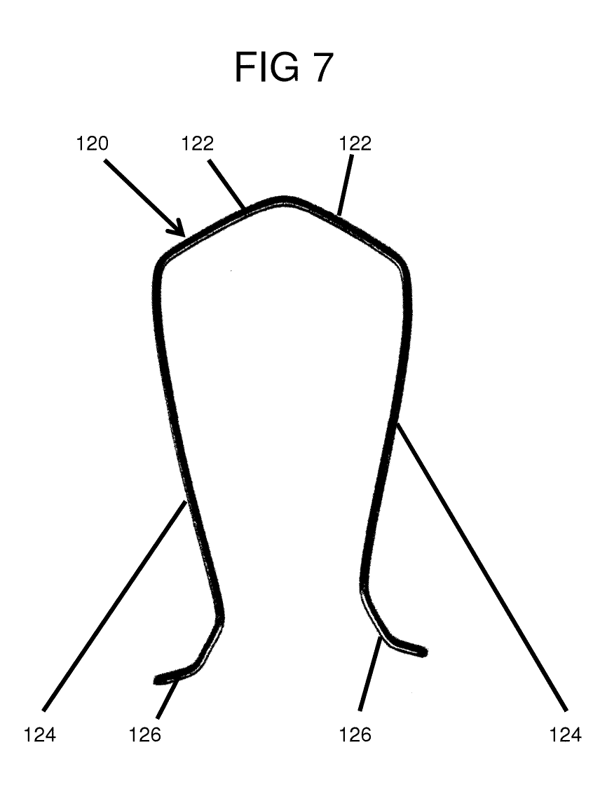

FIG. 7 is a view of a generally dome top spring type tourniquet 120 with segments 122, 124 that are slanted inward with the lower ends 126, forming handles, turned outward to ease in placing the tourniquet 120 over an appendage when spread that when released will spring back toward rest shape to properly compress the appendage to restrict blood flow for a procedure. The tourniquet 120 can be temporarily spread apart to permit blood flow during a procedure and released to recompress the appendage as needed without. Tourniquet 120 can also be removed and replaced as needed during a procedure and removed after completion of the procedure. The tourniquet 120 may also be used to hold the drape or other material in place as needed.

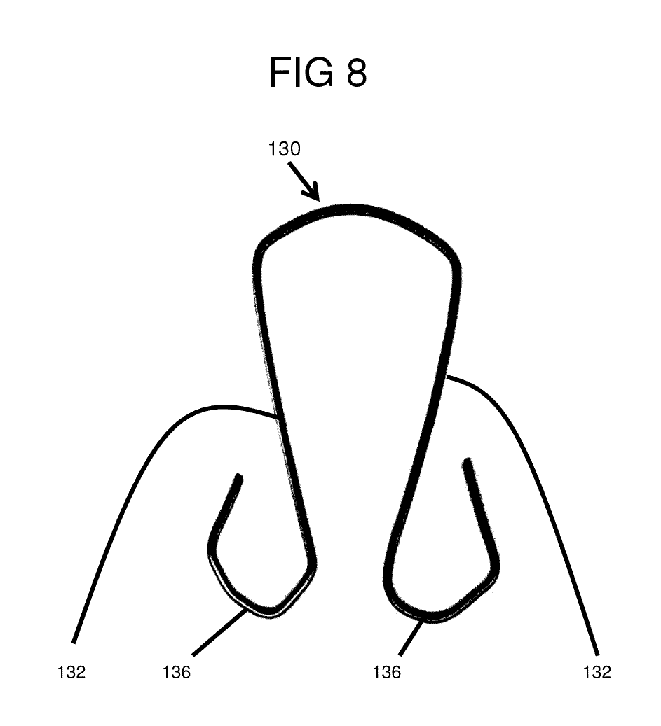

FIG. 8 is a view of a generally rounded top spring type tourniquet 130 with sides that are slanted inward with the lower ends turned outward and upward to form handles 136 to grasp when spreading the open ended tourniquet 130 over an appendage that when released will spring back toward rest shape to properly compress the appendage to restrict blood flow for a procedure. The tourniquet 130 can be temporarily spread apart to permit blood flow during a procedure and released to recompress the appendage as needed. Tourniquet 130 can also be removed and replaced as needed during a procedure and removed immediately after completion of the procedure. The tourniquet 130 may also be used to hold a drape or other material in place as needed.

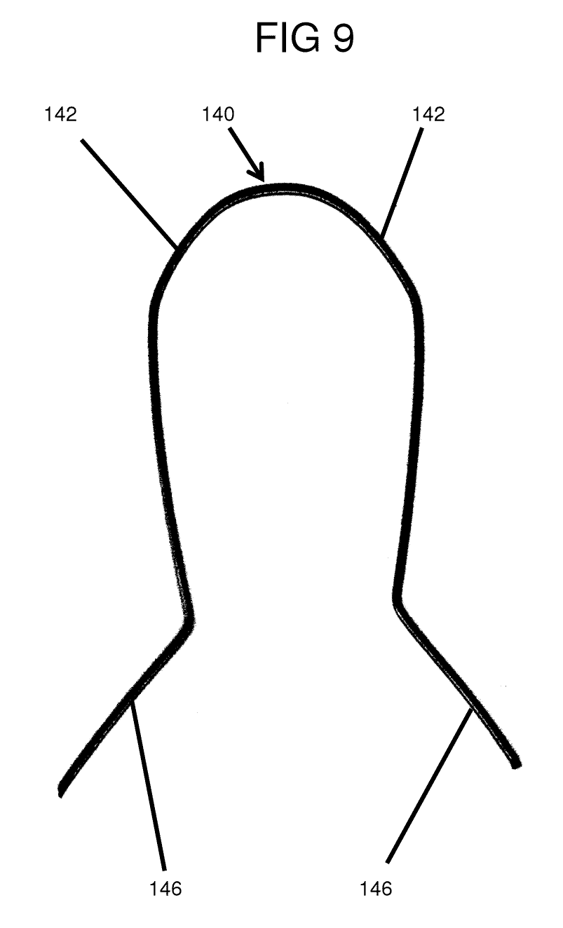

FIG. 9 is a view of a generally rounded top spring type tourniquet 140 with sides 142 that are slanted inward with lower ends 146 slanted outward to form handles to aid when spreading the open ended tourniquet 140 over an appendage that when released will spring back toward rest shape to properly compress the appendage to restrict blood flow for a procedure. The tourniquet 140 can be temporarily spread apart to permit blood flow during a procedure and released to recompress the appendage to restrict blood flow. Tourniquet 140 can also be removed and replaced as needed during a procedure and removed immediately after completion of the procedure. The tourniquet 140 may also be used to hold a drape or other material in place as needed.

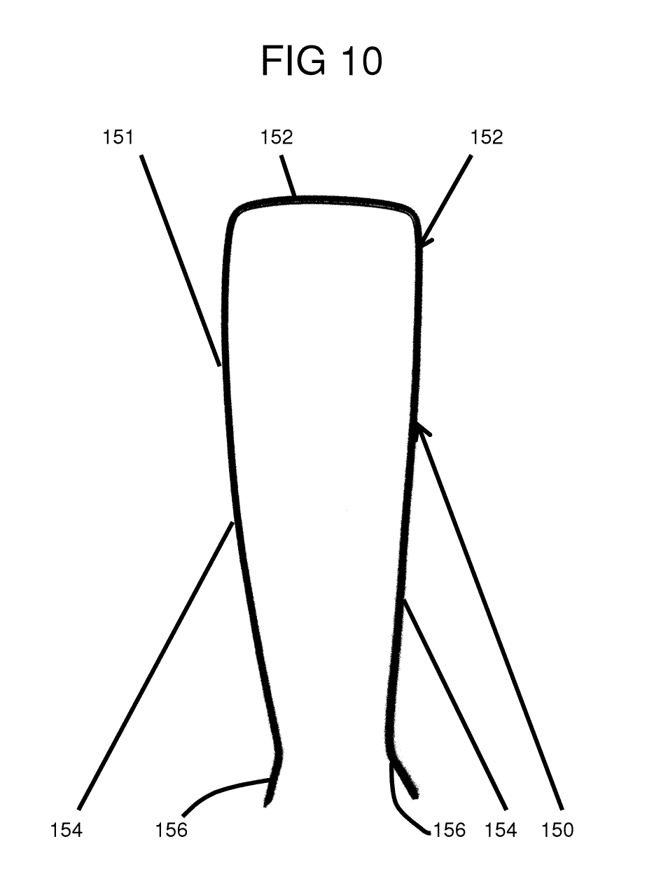

FIG. 10 is a view of a tourniquet 150 with a generally straight top 152 and top side apex with sides 154 that are slanted inward with lower ends 156 that are slanted outward to form handles for spreading the open ended tourniquet 150 over an appendage that when released will spring back toward rest shape to properly compress the appendage to restrict blood flow for a procedure. The tourniquet 150 can be temporarily spread apart to permit blood flow during a procedure and released to recompress the appendage to restrict blood flow as needed. Tourniquet 150 can also be removed and replaced as needed during a procedure and removed immediately after completion of the procedure. The tourniquet 150 may also be used to hold a drape or other material in place as needed.

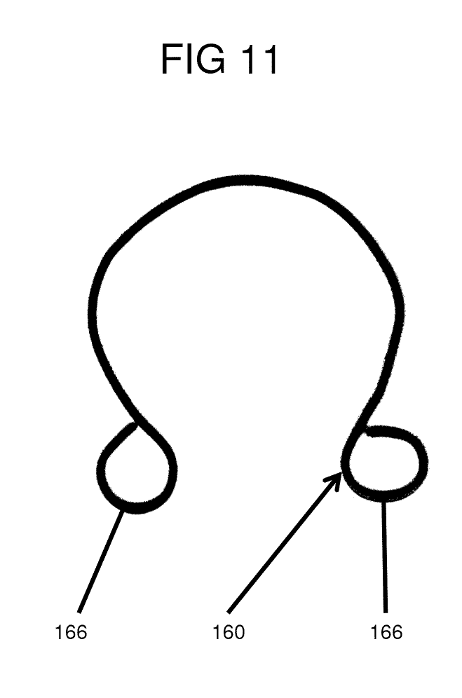

FIG. 11 is a view of a generally circular or semi-circular open ended tourniquet 160 with handles 166 to grasp when spreading the open ended tourniquet 160 over an appendage that when released will spring back toward rest shape to properly compress the appendage to restrict blood flow for a procedure. The tourniquet 160 can be temporarily spread apart to permit blood flow during a procedure and released to recompress the appendage as needed. Tourniquet 160 can also be removed and replaced as needed during a procedure and removed immediately after completion of the procedure. The tourniquet 160 may also be used to hold a drape or other material in place as needed. A semi-circular open ended tourniquet 160 does have certain advantages. It would be relatively easy and inexpensive to manufacture using conventional forming practices. It would also be easy to predict the exact force deflection curve for such a relatively simple structure. This shape should also be more intuitive and more easily understood by the medical professional charged with monitoring the status of the tourniquet.

In use, the technician will grasp the handles or sides of the various embodiments of the tourniquets discussed so far and draw the handles apart to the extent necessary for the tourniquet to pass over the appendage, at which point the tourniquet is allowed to return towards rest shape. As would be realized the extent to which the tourniquet relaxes towards rest shape is dependent on the size of the appendage contained therein. And as would be realized the size of the tourniquet is dependent on the size of the appendage. Each of the embodiments of FIGS. 1-11 function in a similar manner and the various version depict the many detailed forms which can broadly encompass the invention described herein.

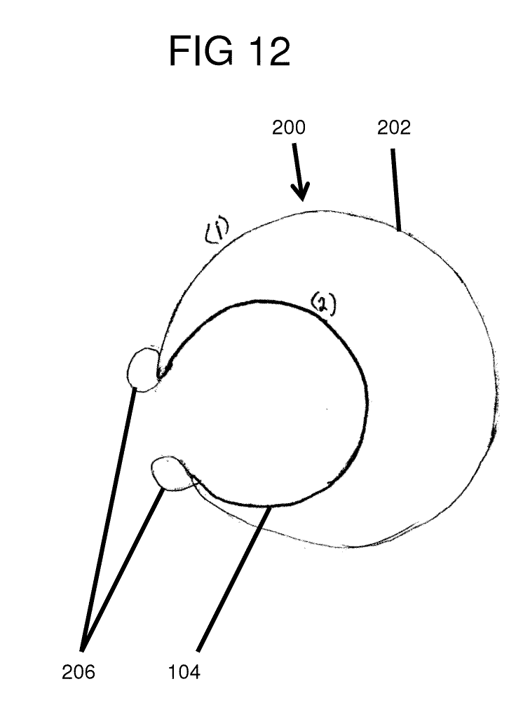

The embodiment of FIG. 12 differs from the previous embodiments in that the tourniquet 200 comprises an outer spring member 202 and an inner flexible fabric member 104 that are joined together adjacent the terminal ends 206 forming handles. The flexible fabric member can comprise a fabric of the type used in conventional tourniquets, and it is this flexible fabric that will engage the patient's arm or appendage about which it will extend. The outer spring member 202 will apply the force and the pressure applied to the appendage. The tourniquet 200 has certain characteristics in common with the semicircular tourniquet 160 shown in FIG. 11. It should be apparent however, that the diameter of the spring member 202 will be greater than the semicircular spring member in tourniquet 160, since spring member 202 will only engage the surface of an appendage at terminal ends 206. Different force characteristics will therefore be exhibited by the spring member 202.

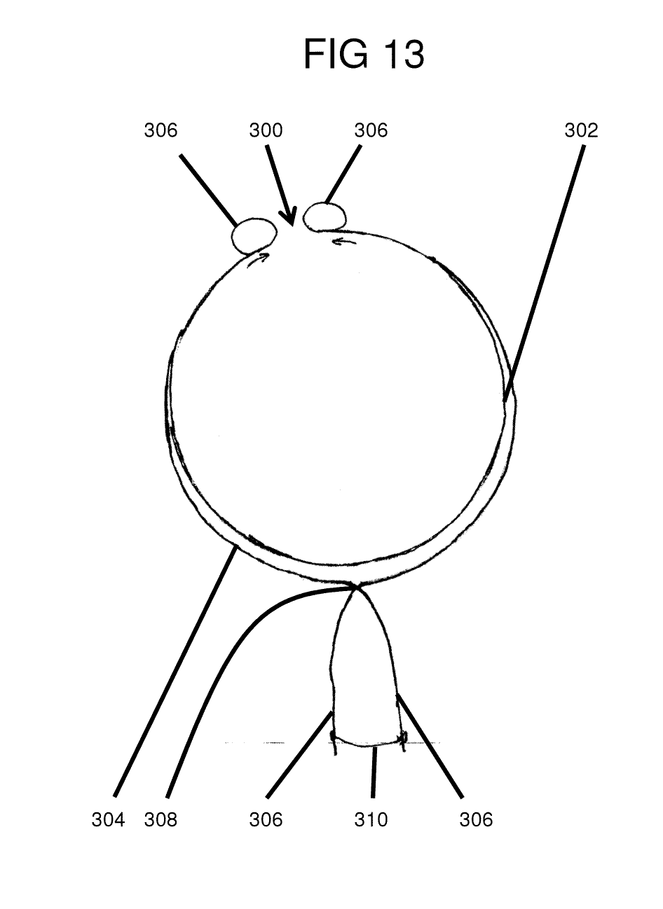

FIG. 13 show a scissor configuration that can be used as a spring biased tourniquet. There are at least four different combinations of devices 300 which could have the shape depicted in FIG. 13.

First if the inner layer 304 is a fabric type material. A coil spring at the pivot point 308 could be configured to bias the scissor handles 306 toward each other so that the tourniquet 300 would normally be in an open position. The operator would press the scissor handles 306 to exert pressure on the appendage and dilate the vein or close off an artery.

The second version of tourniquet 300 would be the same as the first of the alternate versions, but the coil spring at the pivot 308 could be configured to bias the scissor handles 306 away from each other so that the tourniquet section would normally be in a closed position, and pressure would be exerted when the operator releases the handles. The operator could press the handles to cause the tourniquet section to release pressure on the appendage and allow removal of the tourniquet 300.

In a third version of the scissor tourniquet 300 the inner generally circular member 302 in the tourniquet section is a spring member that will be closed in the neutral state. Pressure will be exerted on the appendage when the scissor handles 306 are released. Closing of the handles 306 would open the tourniquet.

In a fourth version of the scissor tourniquet 300 the inner circular member 302 in the tourniquet section is a spring member that will be open in the neutral state. Closing the handles 306 will then close the tourniquet to exert pressure on the appendage. In this configuration, some spring strap or restrain 310 would need to be applied to the scissor handles 306 to keep the handles closed so that pressure would be applied to the patient's appendage.

One procedure for which the spring biased tourniquet of this invention is especially useful is for a percutaneously or peripherally inserted central catheter PICC insertion. In this procedure a PICC is inserted through a vein, typically in the upper arm. The PICC is advanced until the catheter tip is located in a large vein in the chest near the heart to provide intravenous access. Application of a sterile spring biased tourniquet to an appendage between the PICC insertion site and the heart results in venous dilation so that the PICC can be more readily inserted into the chosen vein. This procedure is also especially useful in that it aids in preventing trauma to the vein because tourniquet time is easily controlled. Venous dilation provides a larger entry for the catheter.

The ease with which a sterile spring biased tourniquet of this invention can be positioned on the appendage, such as the upper arm above the insertion site, simplifies this procedure and decreases tourniquet time, which decreases chances of patient problems, without requiring special skill or judgment on the part of the medical practitioner. The opposite ends of the spring biased tourniquet are simply extended away from each other so that the spring biased tourniquet can be inserted over the appendage, and the opposite ends can then be released when the tourniquet is in position. The spring force generated by the tourniquet when the ends are released will cause the vein, below the tourniquet to dilate. In the preferred embodiments depicted herein, handles on the opposite ends of the tourniquet can be grasped to spread the opposite ends of the tourniquet apart, and these handles can be released when the tourniquet is in position. The handles also provide a smooth surface at the ends of the tourniquet. Routinely the tourniquet is used in combination with a surgical drape which is positioned over the patient. Normally the sterile surgical drape is a paper fabric. Traditionally the tourniquet is under the sterile drape for PICC insertion and is difficult to release and extremely difficult to reapply without contaminating the whole procedure. In contrast the preferred embodiment can be applied, adjusted and removed as needed for and during a procedure without compromising sterile technique during PICC insertion. When the smooth handles of the tourniquet are positioned over the surgical drape, the smooth edges will not tear or damage the surgical drape. An alternative method of extending the opposite ends of the tourniquet would be to grasp the tourniquet in the middle using only one hand and push the outer ends apart. With this alternative procedure, the smooth surface formed by the handles on the ends of the tourniquet will also prevent tears or damage to the surgical drape. It follows therefore that a smooth surface on the ends of the spring tourniquet need not be in the form of handles. For example, a smooth spherical member may be attached to each end of the spring biased tourniquet so that the opposite ends will not tear the surgical drape. The handles also need not be in the form of a substantially closed curved section, as depicted in the preferred embodiment. Handles could be in the form of straight sections, each of which may be grasped by the PICC inserter or by a surgical technician. Different configurations at the end of a spring biased tourniquet could be employed for tourniquets intended for use in different situations. For example a spring biased tourniquet suited for use on the upper muscular areas of the leg of a well-conditioned athlete may require a tourniquet having different spring load and deflection characteristics than one suited for use on the arm of a child. Therefore the handles of the shape of the ends of the tourniquet could be altered to fit the particular procedure or patient.

One of the significant characteristics of a sterile spring biased tourniquet for use in a PICC insertion procedure is that a sterilized tourniquet can be used over a sterile drape instead of under a sterile drape. Application of the spring biased tourniquet over the sterile drape enables the health care provider to easily manipulate the tourniquet while simultaneously maintaining sterile technique during the PICC insertion which is a great advantage and improvement. Another significant characteristic is that the tourniquet is intended to be positioned on top of the surgical drape, where it is clearly visible. This helps avoid problems that might result when a surgical drape is positioned over a conventional tourniquet and the conventional tourniquet is not visible. Especially in long or complicated procedures, the medical professionals may be distracted and may not release the pressure imposed by the tourniquet resulting in thrombosis or other injuries to the patient. The spring biased tourniquet of the instant invention is located on the outside of the surgical drape where it is clearly visible and where pressure can be easily relieved during a procedure by adjusting (spreading) the tourniquet. Also the tourniquet can be removed and reapplied as needed and not compromise sterile technique during a procedure. The spring biased tourniquet can also have a shape in which portions of the tourniquet will not follow the contour of the patient's appendage, and will significantly protrude so that it is not only easily accessed but clearly detectable. The spring biased tourniquet of the instant invention can be coated with contrasting colors so that it cannot be ignored or forgotten or suitable labels can be attached to the tourniquet wherein they are clearly visible and separate from the surgical drape with which they are used, but do not interfere with the PICC insertion or other surgical procedure.

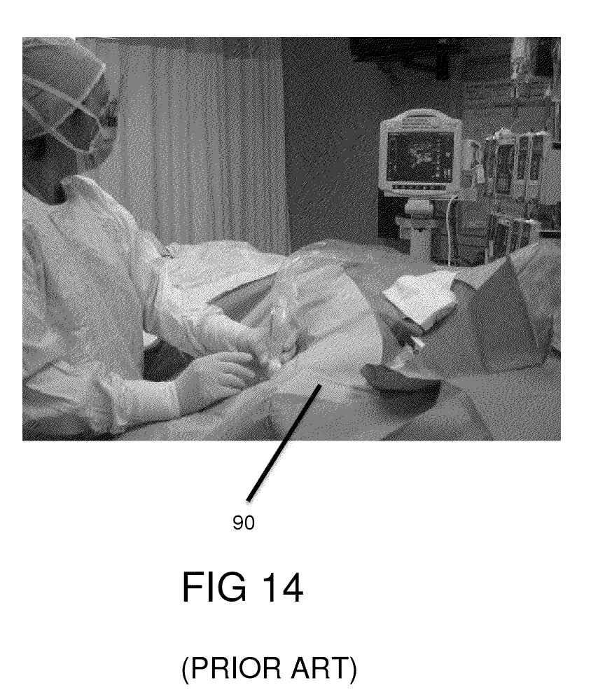

A full body drape 90 of the type commonly employed in a PICC procedure is shown in FIG. 14. Full body drapes are commonly available from suppliers, such as Kimberly Clark. In a procedure in which a PICC is to be inserted through the patient's arm, such a full body drape is commonly employed to establish Maximal Barrier Precautions. To establish such precautions, at least the entire upper body and arm of the patient is draped (while maintaining a sterile field) leaving only a small opening at the insertion site. When this is done according to the prior art practice a tourniquet is applied to the patient's arm above the insertion site, and this prior art tourniquet will be covered by the surgical drape, and thus will not be visible to responsible medical professionals performing or assisting in the procedure. The present invention will allow the tourniquet to be applied on the exterior of the surgical drape, where it is clearly visible and where pressure can be reduced or released without compromising the sterile field.

A Kimberly Clark full body drape is applied after the patient's arm is prepped, by first placing a sterile drape under the patient's arm with the head of a body stamp or profile on the drape aligned with the patient. Adhesive liners are aligned with the prepped area and removed from the drape. A fenestrated area of the drape is placed over the inserting site. The drape is then unfolded laterally on both sides of the patient. The drape is unfolded towards the head and is then pulled over the patient towards the feet for recommended full body coverage. A liner sheet is removed from the fenestrated area, and the appropriate medical professional then firmly presses around the fenestration to seal adhesive to the skin. The drape will then preferably cover substantially the entire body of the patient or at least the upper portion of the body, to form a sterile field excluding the surrounding environment and equipment, such as ultrasonic apparatus, that are used during a PICC insertion procedure. In the prior art, the tourniquet used to provide venous dilation for insertion of the catheter, is beneath the surgical drape. When a tourniquet according to the instant invention is used, the tourniquet can be applied to the arm or other appendage after the surgical drape has been deployed and on the outside of the surgical drape. In this position, the tourniquet of the instant invention is easily attached, removed and pressure can be partially release as required.

The instant invention has been depicted in a number of alternate embodiments. It should be understood that the invention is not limited to these representative embodiments, as the following claims are applicable to structures that may differ in certain details from the representative embodiments depicted herein. Tourniquets according to this invention are also not limited to the common practice of attending to a wound or to use with a PICC as herein described in detail.

We claim:

1. A method of applying a tourniquet to an appendage to at least partially restrict blood flow through the appendage comprising:
providing a tourniquet comprising:
a polygon shaped upper frame comprising:
linear upper frame elements meeting at an upper apex;
linear side frame elements wherein each linear side frame element of said linear side frame elements meets a linear upper frame element of said linear upper frame elements at an upper side apex;
linear lower frame elements wherein each linear lower frame element of said linear lower frame elements meets one said side linear side frame element at a lower side apex wherein each linear lower frame element of said linear lower frame elements each comprises a terminus; and
opposed handles wherein a handle of said opposed handles extends from each terminus in a position to be grasped to outwardly deflect the linear side frame elements, the opposed handles remaining free from each other throughout their entire range of movement;
grasping each handle and persuading said handles away from each other thereby forming a passage between said termini sufficiently large to pass said appendage through said passage;
passing said appendage through said passage; and
allowing said handles to decrease said passage to secure said appendage within the polygon shaped upper frame to at least partially restrict blood flow through the appendage.

2. The method of applying a tourniquet to an appendage of claim 1 wherein said handle comprises linear portions.

3. The method of applying a tourniquet to an appendage of claim 1 further comprising:
providing a surgical drape.

4. The method of applying a tourniquet to an appendage of claim 3 further comprising:
placing said surgical drape on said appendage and securing said surgical drape to said appendage with said tourniquet.

5. A tourniquet comprising:
a generally polygon shaped tourniquet upper frame comprising:
linear tourniquet upper frame elements meeting at an upper apex;
linear tourniquet side frame elements wherein each linear side frame element of said linear side frame elements meets a linear upper frame element of said linear upper frame elements at an upper side apex;
linear tourniquet lower frame elements wherein each linear lower frame element of said linear lower frame elements meets one said side linear side frame element at a lower side apex each linear lower frame element of said linear lower frame elements comprises a terminus; and
opposed handles wherein a handle of said opposed handles is attached to each terminus and opposed handles remain free from each other during their entire range of movement.

6. A tourniquet for use in restricting blood flow through an appendage, the tourniquet comprising:
a member extending from a first terminus to a second terminus at opposite ends of the member, the member exhibiting spring characteristics;
the member defining an upper frame through which the appendage extends when the tourniquet is positioned to restrict blood flow through the appendage;
opposite lower frame elements extending from opposite sides of the upper frame to the first and second terminus at the opposite ends of the member, the opposite lower frame elements extending toward each other from the upper frame toward the first and second terminus, the member being deflectable from a rest shape to a deflected state with the opposite ends of the member forming a passage to allow movement of the appendage through the passage into the upper frame, return of the member toward a rest shape bringing the upper member into engagement with the appendage to restrict blood flow through the appendage.

7. The tourniquet of claim 6 wherein the opposite ends overlap when the member is in a rest shape.

8. The tourniquet of claim 6 wherein the opposite ends are laterally spaced from each other and do not overlap when the member is in a rest shape.

9. The tourniquet of claim 6 wherein the member has a polygon shape.

10. The tourniquet of claim 6 wherein the member has a semi-circular shape.

11. A tourniquet for use dilating a vein in an appendage for inserting a peripherally inserted central venous catheter, the tourniquet comprising:
a spring biased elongate frame member formed to enclose a central opening on at least three sides with handles on a fourth side, the handles being positioned to outwardly deflect portions of the elongate frame member to allow the frame member to receive the appendage through the central opening, the handles also comprising means for controlling pressure applied to the vein by controlling the deflection of the frame member relative to the appendage.

12. The tourniquet of claim 11 wherein the elongate frame member is formed of multiple linear segments, each segment extending at an acute angle relative to an adjacent linear segment.

13. The tourniquet of claim 11 wherein the elongate frame member has a semi-circular shape.

14. The tourniquet of claim 11 wherein the handles overlap when the spring biased member is in a neutral, unbiased position.

15. The tourniquet of claim 11 wherein the handles are laterally spaced and do not overlap when the spring biased member is in a neutral, unbiased position.

16. A method of altering the dilation of a vein in an appendage during a peripherally inserted central venous catheter (PICC) procedure comprising the steps of:
covering at least the appendage with a sterile surgical drape to establish a sterile barrier prior to inserting PICC into the vein in the appendage through an opening in the surgical drape;
applying a spring biased tourniquet around the exterior of the surgical drape and around the appendage to dilate the vein by applying spring pressure to the appendage;
releasing pressure on the vein and the appendage by deflecting the tourniquet against spring force exerted by the spring biased tourniquet without otherwise moving the surgical drape;
whereby the spring biased tourniquet remains visible on the exterior of the surgical drape during the PICC procedure so that pressure can be applied or released on the appendage and the vein without compromising sterile conditions maintained by the surgical drape.
